(19) United States
(12) Patent Application Publication
(10) Pub. No.: US 2017/0181699 A1
(43) Pub. Date: Jun. 29, 2017

(54) SYSTEM AND METHOD FOR PREDICTING CIRCADIAN PHASE

(71) Applicant: KONINKLIJKE PHILIPS N.V., EINDHOVEN (NL)

(72) Inventors: SERVERIUS PETRUS PAULUS PRONK, VUGHT (NL); LUCAAS JOSEF MARIA SCHLANGEN, EINDHOVEN (NL); HENNING MAASS, WAALRE (NL)

(21) Appl. No.: 15/304,868

(22) PCT Filed: Apr. 8, 2015
(86) PCT No.: PCT/IB2015/052555

§ 371 Date: Oct. 18, 2016

Related U.S. Application Data
(60) Provisional application No. 61/983,537, filed on Apr. 24, 2014.

(51) Int. Cl. A61B 5/00; A61B 5/01

(52) U.S. Cl. CPC A61B 5/4857; A61B 5/01; A61B 5/7275

(57) ABSTRACT

Systems and methods to predict circadian phase of a subject use one or more sensors to track light exposure of the subject over a period and determine the current circadian phase. A prediction for future light exposure is used to predict a future circadian phase.

### BACKGROUND

1. Field

The present disclosure pertains to a system and method for predicting the circadian phase of a subject, and, in particular, to using a prediction of future light exposure to predict future circadian phase.

2. Description of the Related Art

The impingement of radiation on subjects to impact the circadian rhythms and/or to address light deficient disorders may be known. Generally, these treatments involve shining light directly towards a patient's eyes, e.g. while the patient is awake, to alleviate or cure light deficient disorders including, but not limited to, Seasonal Affective Disorder (SAD), circadian sleep disorders and circadian disruptions associated with, e.g., jet-lag, shift-work, and/or other occupational conditions that may cause circadian disruptions.

Various models may be used to calculate and/or estimate a subject's circadian phase. Typically, calculations and/or estimations are made based on light exposure and/or other measurements during a preceding period, e.g. of one or more days. Once the circadian phase of a particular subject has been determined, it may be adjusted as desired and/or recommended by, e.g., light therapy.

There are various types of light therapy devices presently available. One type of device is large in size and floor or desk mountable. These devices include light sources of fluorescent bulbs or large arrays of light emitting diodes. Although they can be moved from one position to another, they are not generally portable and require a scheduled time period of being stationary during the active part of the day. In addition, the light source is quite fragile. One type of light therapy device may be head mountable. These devices may form eyeglasses or visors. These devices generally lack features that enable them to be used while functioning during sleep.

### SUMMARY

Accordingly, it is an object of one or more embodiments of the present invention to provide a system to predict circadian phase of a subject. The system includes one or more sensors and one or more physical processors. The one or more sensors are configured to generate output signals conveying information related to light exposure of the subject. The one or more physical processors are configured to determine one or more light exposure parameters based on the generated output signal; determine a current circadian phase of the subject based on the one or more light exposure parameters; predict future light exposure for a first future period based on the generated output signals for a preceding period; and predict a future circadian phase based on the current circadian phase and the predicted future light exposure.

It is yet another aspect of one or more embodiments of the present invention to provide a method to predict circadian phase of a subject. The method includes generating output signals conveying information related to light exposure of the subject; determining one or more light exposure parameters based on the generated output signal; determining a current circadian phase of the subject based on the one or more light exposure parameters; predicting future light exposure for a first future period based on the generated output signals for a preceding period; and predicting a future circadian phase based on the current circadian phase and the predicted future light exposure.

It is yet another aspect of one or more embodiments to provide a system configured to predict circadian phase of a subject. The system includes means for generating output signals conveying information related to light exposure of the subject; means for determining one or more light exposure parameters based on the generated output signal; means determining a current circadian phase of the subject based on the one or more light exposure parameters; means for predicting future light exposure for a first future period based on the generated output signals for a preceding period; and means for predicting a future circadian phase based on the current circadian phase and the predicted future light exposure.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 schematically illustrates a system configured to predict circadian phase of a subject, in accordance with one or more embodiments;

FIG. 2 illustrates a method to predict circadian phase of a subject, according to one or more embodiments.

FIG. 3A illustrates a graph depicting the circadian rhythm over time.

FIG. 3B illustrates a graph depicting light exposure over time.

FIG. 4A illustrates a graph depicting measured and predicted light exposure of time.

FIGS. 4B-4C illustrates phase response curves for particular exposure intensities according to a particular model for circadian phase determination.

FIGS. 5A-5B illustrates graphs depicting mean and standard deviation of the error in circadian phase estimation based on varying preceding periods (the periods being used to determine future light exposure).

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other. As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality). As used herein, the term “include” shall be used inclusively to mean any item of a list, by example and without limitation, and/or any combination of items in that list, to the extent possible.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

Mammalian circadian systems coordinate the timing of an animal's physiological and behavioral functions with local position on the planet. The circadian system depends primarily upon the 24-hour light-dark pattern incident on the retinae. The phototransduction mechanisms responsible for human circadian phototransduction have been elucidated well enough that devices may take advantage of this understanding and adjust circadian timing as desired.

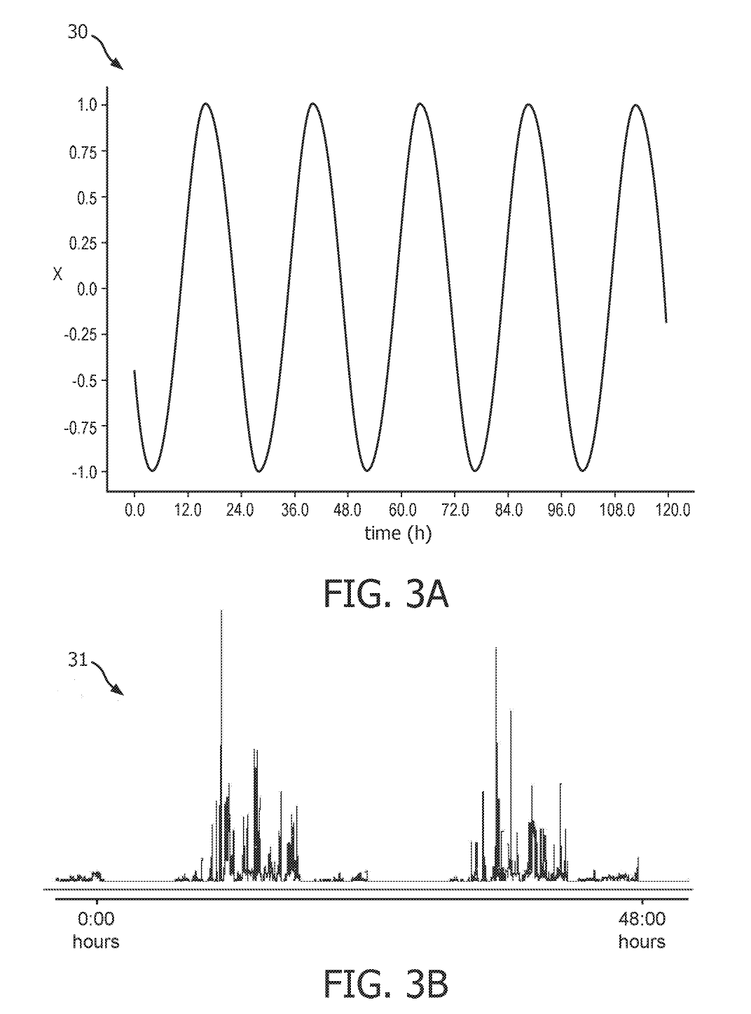

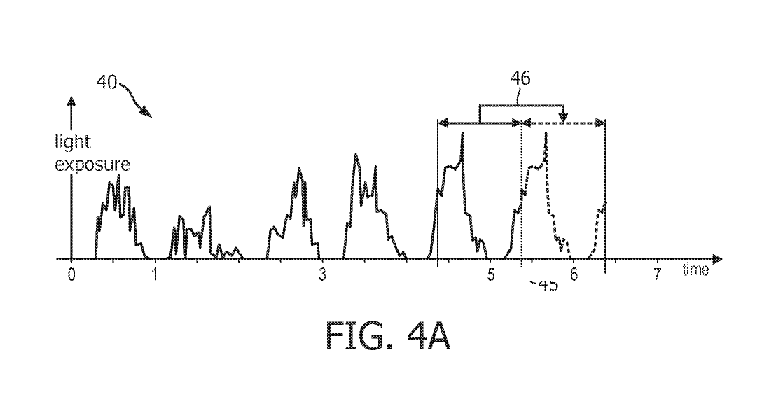

Various biomarkers may serve to establish a particular moment in the circadian process, which may be referred to as circadian phase. For example, the time at which the core body temperature attains a minimum value may be a biomarker. This time or moment may be referred to as core body temperature minimum or CBTmin. As used herein, the term CBTmin may be used to indicate the value of the minimum core body temperature or the moment of its attainment, depending on the context of the reference. In some embodiments, CBTmin is used as the zero phase (of the circadian rhythm), the zero-point of the circadian phase, or a circadian phase having value zero. Also the moment at which melatonin production starts, under dim-light conditions—is a so-called biomarker. This moment may be referred to as dim-light melatonin onset or DLMO. In some embodiments, DLMO is used to denote circadian phase. For example, under certain light conditions, DLMO occurs at around 22:30 h. By way of illustration, FIG. 3A illustrates a graph 30 depicting the core body temperature fluctuation over time—normalized between minus 1 and positive 1—of a theoretical subject, the graph spanning about 120 hours. As depicted, the circadian phase is grossly cyclical, repeating itself about every 24 hours.

Models that may be used to calculate and/or estimate a subject's circadian phase may use one or more of the following information as input: light exposure during a preceding period, CBTmin, DLMO, subject-specific parameters, sleep-wake information, and/or other types of information. Note that light may shift the circadian phase of a subject, depending on, at least, the intensity of the light and the relative timing of light exposure with respect to the current circadian phase. In some models, one or more types of information may be used, for example in combination, to determine an expected shift of the circadian phase. For example, light administered after the CBTmin (typically early in the morning for a properly aligned circadian rhythm) may advance the circadian phase, whereas light administered before the CBTmin (typically in the evening or early in the night for a properly aligned circadian rhythm) may delay the circadian phase.

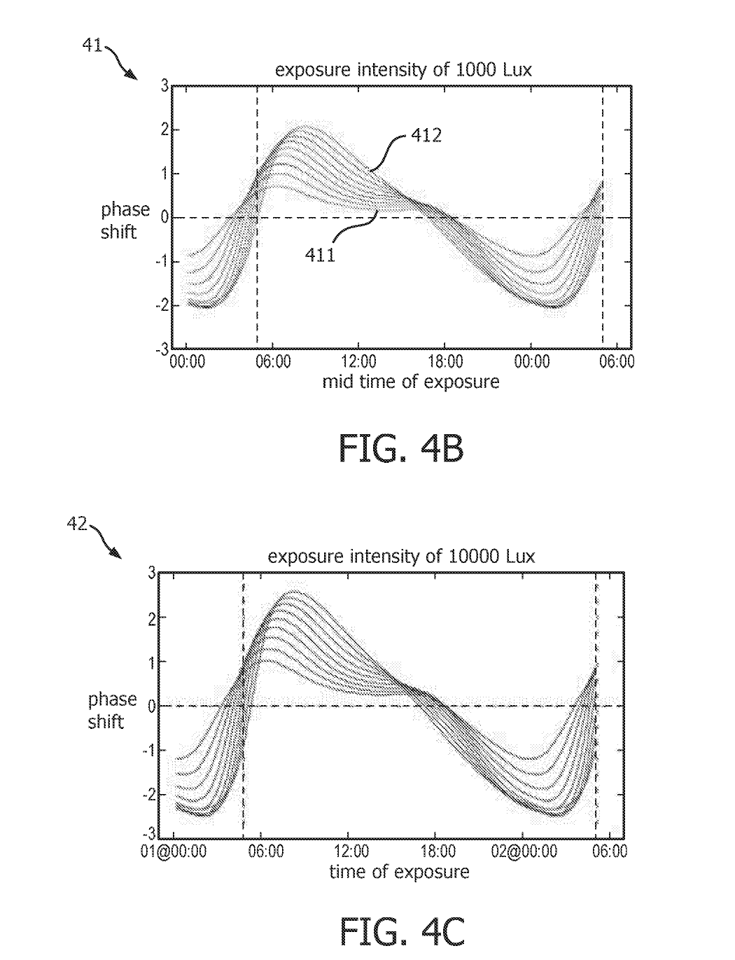

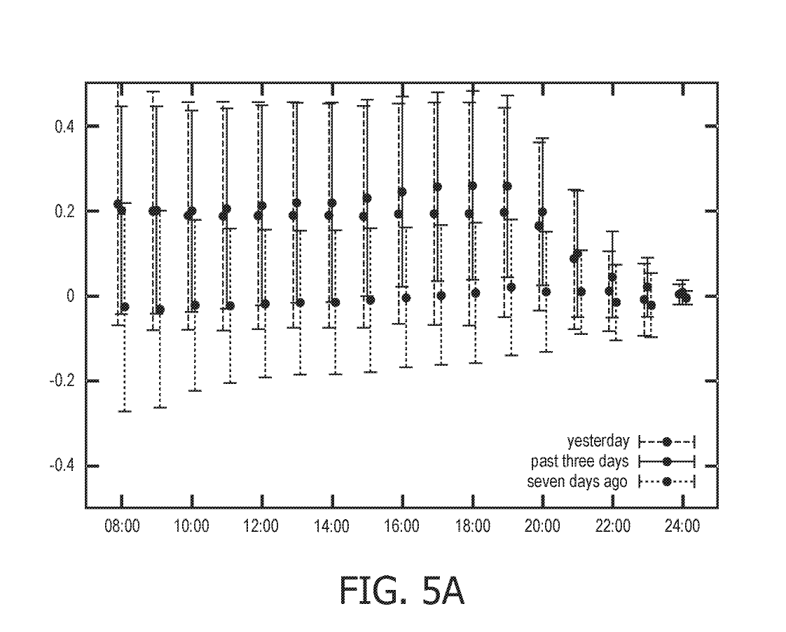

By way of illustration, FIG. 4B illustrates phase response curves 41 for a light exposure intensity of 1000 Lux during a period spanning about 30 hours, which may be used for modeling purposes and/or other (theoretical) purposes. Phase response curves 41 include nine curves, ranging from a one-hour exposure duration indicated by a phase response curve 411 to a nine-hour exposure duration indicated by a phase response curve 412. The dotted vertical lines indicate two successive CBTmin biomarkers, occurring at about 4:30 am. The Y-axis denotes the amount of circadian phase shift, ranging between about minus two and positive two hours. For example, a one-hour exposure to light having a 1000 Lux light exposure intensity, administered at 6:00 AM may shift the circadian phase by about 40 minutes (i.e. advance the circadian phase, whereas the same exposure administered at about 6:00 PM has hardly any effect, and the same exposure administered at about 1:00 AM may shift the circadian phase by about minus forty minutes (i.e. delay the circadian phase). Likewise, a nine-hour exposure to light having a 1000 Lux light exposure intensity, administered at about 8:00 AM may shift the circadian phase by about two hours, whereas the same exposure administered at about 5:00 PM has hardly any effect, and the same exposure administered at about 1:00 AM may shift the circadian phase by about minus two hours (i.e. a delay of the circadian phase). Further by way of illustration, FIG. 4C illustrates phase response curves 42 for a light exposure intensity of 10000 Lux during a period spanning about 30 hours. Phase response curves 42 in FIG. 4C illustrate a generally larger response than phase response curves 41 in FIG. 4B, by virtue of administering a higher light exposure intensity.

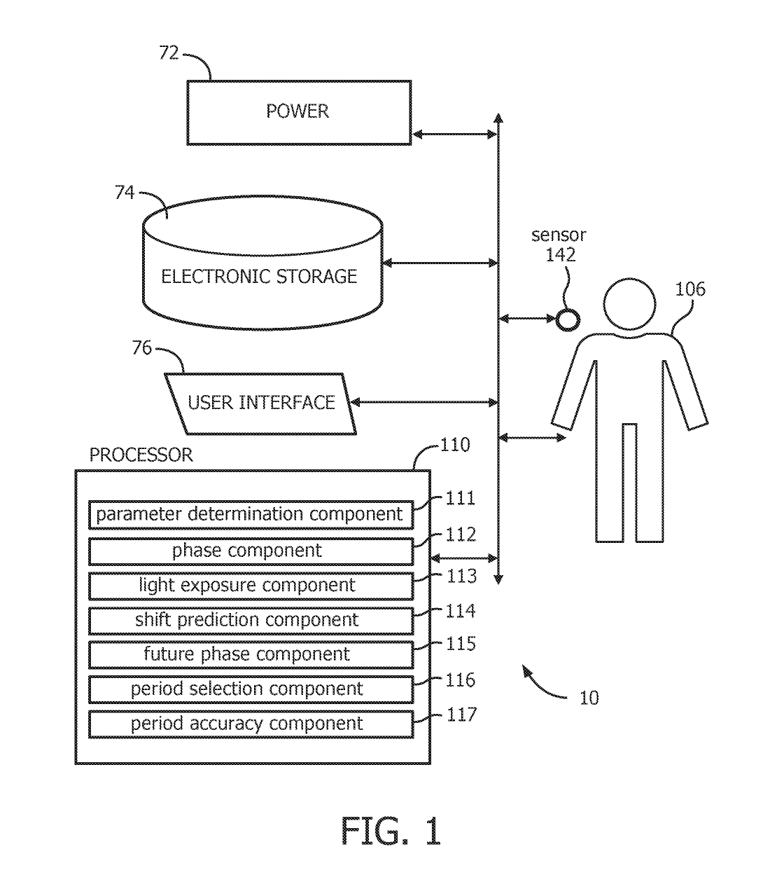

FIG. 1 illustrates a system 10 configured to predict circadian phase of a subject 106, in accordance with one or more embodiments. Predicted circadian phase may be used to treat, alleviate, and/or cure light deficient disorders including Seasonal Affective Disorder (SAD), Delayed Sleep Phase Syndrome (DSPS), Advanced Sleep Phase Syndrome (ASPS), circadian disorders, and/or circadian disruptions associated with, e.g., jet-lag and/or shift-work. In some embodiments, system 10 may be configured to predict and/or modify a characteristic of the circadian rhythm of subject 106, including but not limited to the phase of the circadian rhythm.

System 10 may include one or more of a power source 72, one or more sensors 142, one or more physical processors 110, various computer program components, electronic storage 74, a user interface 76, and/or other components. The computer program components may include a parameter determination component 111, a phase component 112, a light exposure component 113, a shift prediction component 114, a future phase component 115, a period selection component 116, a period accuracy component 117, and/or other components.

One or more sensors 142 of system 10 in FIG. 1 may be configured to generate output signals conveying information related to light exposure of subject 106, physiological, environmental, and/or patient-specific (medical) parameters related to subject 106, and/or other information. System 10 may use any of the generated output signals to monitor subject 106. In some embodiments, the conveyed information may be related to parameters associated with the state and/or condition of subject 106, motion of subject 106, wakefulness and/or sleep state of subject 106, the breathing of subject 106, the gas breathed by subject 106, the heart rate of subject 106, the respiratory rate of subject 106, vital signs of subject 106, including one or more temperatures, oxygen saturation of arterial blood (SpO₂), whether peripheral or central, and/or other parameters.

In some embodiments, one or more sensors 142 may generate output signals conveying information related to a location of subject 106, e.g. through stereoscopy. The location may be a three-dimensional location of subject 106, a two-dimensional location of subject 106, a location of a specific body part of subject 106 (e.g., eyes, arms, legs, a face, a head, a forehead, and/or other anatomical parts of subject 106), the posture of subject 106, the orientation of subject 106 or one or more anatomical parts of subject 106, and/or other locations.

Sensors 142 may include one or more of a light sensor, an optical sensor, a temperature sensor, a pressure sensor, a weight sensor, an electromagnetic (EM) sensor, an infra-red (IR) sensor, a microphone, a transducer, a still-image camera, a video camera, and/or other sensors and combinations thereof.

The illustration of sensor 142 including one member in FIG. 1 is not intended to be limiting. System 10 may include one or more sensors. The illustration of a particular symbol or icon for sensor 142 in FIG. 1 is exemplary and not intended to be limiting in any way. Resulting signals or information from one or more sensors 142 may be transmitted to processor 110, user interface 76, electronic storage 74, and/or other components of system 10. This transmission can be wired and/or wireless.

One or more sensors 142 may be configured to generate output signals in an ongoing manner, e.g. throughout the day, week, month, and/or years. This may include generating signals intermittently, periodically (e.g. at a sampling rate), continuously, continually, at varying intervals, and/or in other ways that are ongoing during at least a portion of period of a day, week, month, or other duration. The sampling rate may be about 0.001 second, 0.01 second, 0.1 second, 1 second, about 10 seconds, about 1 minute, and/or other sampling rates. It is noted that multiple individual sensors may operate using different sampling rates, as appropriate for the particular output signals and/or (frequencies related to particular) parameters derived therefrom. For example, in some embodiments, the generated output signals may be considered as a vector of output signals, such that a vector includes multiple samples of information conveyed related to one or more parameters of subject 106. Different parameters may be related to different vectors. A particular parameter determined in an ongoing manner from a vector of output signals may be considered as a vector of that particular parameter.

Physical processor 110 (interchangeably referred to herein as processor 110) is configured to provide information processing and/or system control capabilities in system 10. As such, processor 110 may include one or more of a digital processor, an analog processor, a digital circuit designed to process information, an analog circuit designed to process information, and/or other mechanisms for electronically processing information. In order to provide the functionality attributed to processor 110 herein, processor 110 may execute one or more components. The one or more components may be implemented in software; hardware; firmware; some combination of software, hardware, and/or firmware; and/or otherwise implemented. Although processor 110 is shown in FIG. 1 as a single entity, this is for illustrative purposes only. In some implementations, processor 110 may include a plurality of processing units. These processing units may be physically located within the same device, or processor 110 may represent processing functionality of a plurality of devices operating in coordination.

As is shown in FIG. 1, processor 110 is configured to execute one or more computer program components. The one or more computer program components include one or more of parameter determination component 111, phase component 112, light exposure component 113, shift prediction component 114, future phase component 115, period selection component 116, period accuracy component 117, and/or other components. Processor 110 may be configured to execute components 111-117 by software; hardware; firmware; some combination of software, hardware, and/or firmware; and/or other mechanisms for configuring processing capabilities on processor 110.

It should be appreciated that although components 111-117 are illustrated in FIG. 1 as being co-located within a single processing unit, in implementations in which processor 110 includes multiple processing units, one or more of components 111-117 may be located remotely from the other components. The description of the functionality provided by the different components 111-117 described below is for illustrative purposes, and is not intended to be limiting, as any of components 111-117 may provide more or less functionality than is described. For example, one or more of components 111-117 may be eliminated, and some or all of its functionality may be provided by other ones of components 111-117. Note that processor 110 may be configured to execute one or more additional components that may perform some or all of the functionality attributed below to one of components 111-117.

As used herein, the term “determine” and derivatives thereof include one or more of determine, estimate, approximate, measure, and/or any combination thereof. For example, “determining a parameter” may be interpreted as “determining, estimating, approximating, and/or measuring a parameter, as well as any combination thereof.”

Parameter determination component 111 of system 10, depicted in FIG. 1, may be configured to determine one or more of the following types of parameters: light exposure parameters, status parameters, medical parameters, and/or other parameters from output signals generated by one or more sensors 142. Parameters may be related to a subject's physiological, environmental, and/or patient-specific parameters. One or more medical parameters may be related to monitored vital signs of subject 106, and/or other medical parameters of subject 106. For example, one or more medical parameters may be related to whether subject 106 is awake or asleep, or, in particular, what the current sleep stage of subject 106 is. Other parameters may be related to the environment near system 10 and/or near subject 106, such as, e.g., air temperature, ambient noise level, ambient light level, and/or other environmental parameters. One or more physiological parameters may be related to and/or derived from electro-encephalogram (EEG) measurements, electromyogram (EMG) measurements, respiration measurements, cardiovascular measurements, heart-rate-variability (HRV) measurements, autonomic nervous system (ANS) measurements, and/or other measurements. Some or all of this functionality may be incorporated or integrated into other computer program components of processor 110.

In some embodiments, parameter determination component 111 may be configured to determine, track, and/or monitor one or more parameters during a period spanning minutes, hours, days, and/or weeks. For example, in some embodiments, parameter determination component 111 may be configured to determine a light exposure parameter, based on output signals generated by one or more sensors 142, during a period spanning at least a day, and/or intermittently, periodically (e.g. at a sampling rate), continuously, continually, at varying intervals, and/or in other ways that are ongoing during at least a period of a day, week, month, or other duration. For example, parameter determination component 111 may be configured to determine a vector of light exposure parameters.

Phase component 112 may be configured to determine circadian phases of subjects. For example, phase component 112 may be configured to determine a current circadian phase of subject 106. In some embodiments, operation of phase component 112 may be based on a model for circadian phase, including, but not limited to the models described herein that involve core body temperature (e.g. CBTmin), dim-light melatonin onset (DLMO), and/or other biomarkers, physiological parameters, and/or environmental parameters. Alternatively, and/or simultaneously, operation of phase component 112 may be based on one or more parameters, including but not limited to parameters predicted and/or determined by parameter determination component 111. For example, operation of phase component 112 may be based on one or more light exposure parameters. For example, phase component 112 may be configured to determine a current circadian phase of subject 106 based on one or more light exposure parameters for a preceding period spanning one or more days (e.g. indicating light exposure intensity during the preceding period). By way of illustration, FIG. 3B illustrates a graph 31 depicting light exposure of a particular subject over time, as may have been measured during a period spanning about 48 hours. The Y-axis depicts light exposure. In this disclosure, light exposure may interchangeably be referred to as light exposure intensity, light intensity, or illuminance. Models that may be used to calculate and/or estimate a subject's circadian phase may use the information from graph 31 and determine the circadian rhythm of the particular subject, and thus the circadian phase at any time during the period of measurement.

Light exposure component 113 may be configured to determine and/or predict future light exposure. In some embodiments, determinations and/or predictions may pertain to one or more particular period. For example, a particular future time period may include about 8, 12, 16, 20, 24, and/or another numbers of hours from now. In some embodiments, light exposure component 113 may be configured to predict future light exposure for a future period spanning about 24 hours. In some embodiments, the duration of the future period may be variable. In some embodiments, the predicted future light exposure for the next 24 hours may be a fixed pattern and/or profile of light exposure, for example based on standard light exposure in a particular area, or for people having similar characteristics as the particular subject.

In some embodiments, a first prediction may be made at a first moment in time, and may include a future period spanning about 24 hours. A second prediction may be made at a second moment in time that occurs, say, four hours after the first moment in time. In some embodiments, light exposure measurements for the four hours between the first moment and the second moment may be taken into account. The second prediction may include a future period spanning about 20 hours, such that the first prediction and the second prediction roughly cover a future period up to the same moment relative to external or real-world time. In some embodiments, light exposure component 113 may be configured to repeatedly, intermittently, periodically (e.g. at a sampling rate of one hour), continuously, continually, at varying intervals, and/or in other ways that are ongoing predict and/or determine future light exposures.

In some embodiments, determinations and/or predictions by light exposure component 113 may be based on (interpolations of) a light exposure parameter determined by parameter determination component 111. For example, a determination or prediction for a particular future period of 24 hours may be based on the light exposure parameter of the preceding 24 hours. In some embodiments, a determination or prediction for a particular future period may be based on the preceding day, the preceding two days, the preceding three days, the preceding four days, the preceding five days, the preceding six days, the preceding seven days, and/or another number of preceding days.

By way of illustration, FIG. 4A illustrates a graph 40 depicting measured and predicted light exposure over time, spanning about 5 days of measure light exposure, and 1 day of predicted light exposure. At a moment in time 45, roughly at about 9:00 am of the fifth day, the light exposure measured during a preceding period may be used to predict the light exposure for a future period, e.g. for about the next 24 hours. As indicated by arrow 46, in some embodiments, the prediction may be a copy of the light exposure measured during the preceding 24 hours. In some embodiments, the prediction may be based on the preceding 48 hours, 72 hours, 168 hours, 14 days, 21, days, 28 days, and/or another suitable number of hours or days. Predictions may be aggregated and/or averaged light exposure measurements during the preceding hours in various ways. For example, light exposure measurements from more recent days may carry more weight in a prediction than older light exposure measurements. In some embodiments, a weighting function may be used that indicates what weight to attribute to each of the preceding 7, 10, or 14 days. For example, the same day of the week, measured a week ago, may carry more weight than the measured light exposure parameters for four days ago.

Alternatively, and/or simultaneously, a subject's schedule may distinguish work days (or week days) from non-work days (or the weekend), and use this information to determine the weighting function. For example, expected/predicted light exposure for a Saturday may be more similar to a previous Saturday than to the preceding Friday.

Alternatively, and/or simultaneously, a determination or prediction for a particular future period may be based on seasonal information, dusk and dawn predictions, geographical information, global positioning information, weather forecasts and/or predictions, subject-specific travel plans, subject-specific calendar information, and/or other information. For example, a subject's geographical location may affect the predicted future light exposure. For example, a planned meeting (and/or the location thereof) may affect the predicted future light exposure. For example, a planned car-trip, train-ride, or flight may affect the predicted future light exposure.

In some embodiments, light exposure component 113 may be configured to determine and/or predict a light exposure parameter (or vector thereof) repeatedly throughout a particular period. For example, as depicted in FIG. 4A, the first prediction for a particular day may predict a vector of light exposure parameters spanning a first future period of about 24 hours. Every hour (or any other suitable interval) after the first prediction, a new prediction may predict one or more light exposure parameters for either a future period of about 24 hours, or for whatever time remains of the first future period used for the first prediction. New predictions may use measured light exposure for the time that has passed since the first prediction to improve prediction accuracy. The first future period of about 24 hours gradually recedes into the past as the progress of time catches up to the prediction.

In some embodiments, shift prediction component 114 may be configured to determine and/or predict an evolution and/or shift of the circadian process and/or of the circadian phase of subject 106, e.g. with respect to another determined circadian phase such as the current circadian phase of subject 106. Shift prediction component 114 operates in a similar manner as phase component 112, except that the one or more light exposure parameters (or the vector of a light exposure parameter) reflect predicted future light exposure rather than measured light exposure in a preceding period. Future light exposure, e.g. as determined and/or predicted by light exposure component 113, may be combined with the current circadian phase of subject 106 (e.g. as determined by phase component 112) and/or any of the models described herein, to determine an evolution of the circadian process of subject 106. Accuracy of this determination/prediction may be limited to the accuracy of the determination of the current circadian phase and the measured and/or predicted light exposure.

Future phase component 115 may be configured to determine and/or predict a future circadian phase of subject 106. In some embodiments, determination and/or prediction of a future circadian phase may be for a specific future point in time, and/or for a specific future period. For example, the specific future period may be a period spanning one or more hours. In some embodiments, a determination and/or prediction may be based on the determination of the current circadian phase, the measured and/or predicted light exposure, and/or the determined and/or predicted shift of circadian phase (e.g. by shift prediction component 114). For example, the next CBTmin may be predicted to be 24 hours from the previous CBTmin.

For example, subject 106 may plan to participate in a particular future event (e.g. a surgery, a meeting, etc.) the day after tomorrow, scheduled from, e.g., 3:00 pm to 5:00 pm. Future phase component 115 may be configured to predict the future circadian phase of subject 106 during the particular future event. Based on information determined by virtue of this disclosure, light therapy and/or other therapy may be used to adjust one or more parameters of the circadian rhythm of subject 106 in a desired manner.

Period selection component 116 may be configured to select and/or determine a preceding period to be used as a basis for a determination and/or prediction of future light exposure. As described in relation to light exposure component 113 and FIG. 4A, light exposure measurements spanning a preceding period may be used. In some embodiments, the preceding period may be variable. In some embodiments, the preceding period may be selected between about 24 hours and about two weeks. Selection of a preceding period may depend on the day of the week. For example, on Monday, the preceding period may be based on the preceding Monday-to-Friday period. For example, on Tuesday, the preceding period may be based on Monday. For example, on Wednesday, the preceding period may be based on Monday and Tuesday. For example, on Thursday, the preceding period may be based on the period from Monday to Wednesday. When light exposure is expected to change during the weekend, the preceding period used for a Saturday may be the previous weekend.

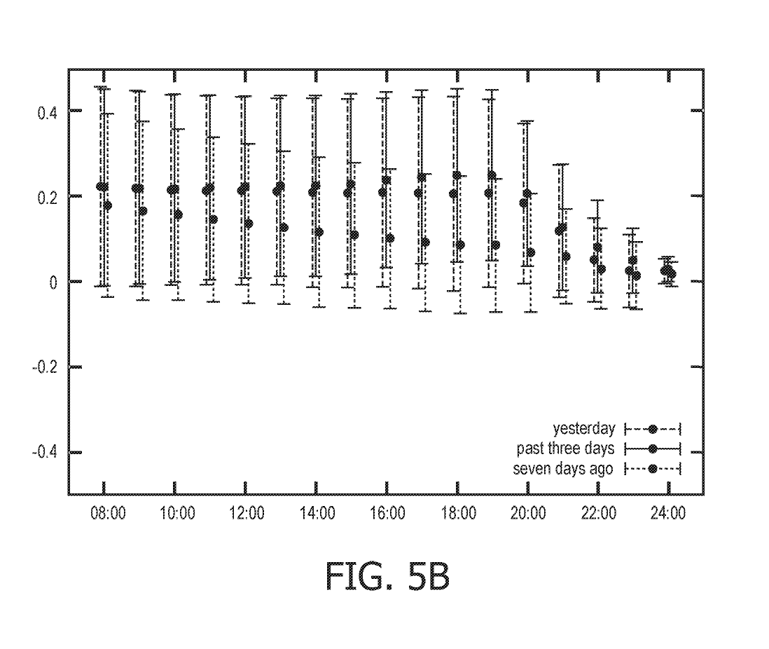

By way of illustration, FIG. 5A illustrates graphs depicting means and standard deviations of the errors in circadian phase estimation based on using varying preceding periods to determine future light exposure. The errors may be determined in hind-sight, after light exposure measurements for, e.g., an entire week have been gathered. The first prediction of a particular day occurs at approximately 8:00 AM. The first set of means and standard deviations (of the error between predicted and actual circadian phase) corresponds to a preceding period of the preceding 24 hours. As the day progresses, the mean and standard deviation of the error become smaller. The second set of means and standard deviations corresponds to a preceding period of the preceding 3 days. As the day progresses, the mean and standard deviation of the error become smaller. Note that the error using a 1-day preceding period is generally larger than the error using a 3-day preceding period. The third set of means and standard deviations corresponds to a preceding period of the preceding 7 days. As the day progresses, the mean and standard deviation of the error become smaller. Note that the error using a 7-day preceding period is generally smaller than the errors using a 1-day or 3-day preceding period. In other words, FIG. 5A reflects that predicting a future light exposure based on light exposure measurements spanning different or more preceding days may result in more accurate predictions for the circadian phase of a particular subject. The use of 1-day, 3-day, and 7-day periods is exemplary and not intended to be limiting in any way.

In some embodiments, period selection component 116 may be configured to select a preceding period from a set of options. In some embodiments, the set of options includes 1-day, 2-day, 3-day, 4-day, 5-day, 6-day, and 7-day periods. In some embodiments, the set of options may include any subset of the preceding 7 days (or 14 days, or 28 days) as options, such that one or more days may be skipped. In other words, the preceding period need not be contiguous.

In some embodiments, operation of period selection component 116 may be based on seasonal information, dusk and dawn predictions, geographical information, global positioning information, weather forecasts and/or predictions, subject-specific travel plans, subject-specific calendar information, and/or other information.

Period accuracy component 117 may be configured to determine the accuracy of a particular preceding period of light exposure measurements as a basis for a prediction of circadian phase of subjects. Period accuracy component 117 may be configured to make determinations for multiple preceding periods from a set of preceding periods, including but not limited to a set of options of preceding periods as used and described in relation to the operation of period selection component 116.

Period accuracy component 117 may be configured to compare accuracies for multiple preceding periods of light exposure measurements as a basis for a prediction of circadian phase, for example for subject 106. Period accuracy component 117 may be configured to determine the most accurate preceding period selected from a set of options. In some embodiments, the accuracy of a particular preceding period may vary over time, for example throughout a week or month. For example, accuracy for Mondays may be highest when using a first preceding period as the basis for aggregating light exposure measurements (using a first particular weighting function spanning the first preceding period). For example, accuracy for Tuesdays may be highest when using a second preceding period as the basis for aggregating light exposure measurements (using a second particular weighting function spanning the second preceding period). The first preceding period may differ from the second preceding period. The first particular weighting function may differ from the second particular weighting function. For example, accuracy for Saturdays may be highest when using a third preceding period as the basis for aggregating light exposure measurements (using a third particular weighting function spanning the third preceding period). The third preceding period may differ from the first and second preceding periods. The third particular weighting function may differ from the first and second particular weighting functions. For example, accuracy for Sundays may be highest when using a fourth preceding period as the basis for aggregating light exposure measurements (using a fourth particular weighting function spanning the fourth preceding period). The fourth preceding period may differ from the first, second, and third preceding periods. The fourth particular weighting function may differ from the first, second, and third particular weighting functions.

In some embodiments, operation of period accuracy component 117 may be based on seasonal information, dusk and dawn predictions, geographical information, global positioning information, weather forecasts and/or predictions, subject-specific travel plans, subject-specific calendar information, and/or other information.

In some embodiments, light exposure component 113, shift prediction component 114, and/or other components of system 10 may be configured such that predictions throughout a day, based on a selected light exposure prediction, are interpolated with the estimation of the previous circadian phase and/or CBTmin (based on light exposure measurements) to produce an improved circadian phase prediction, the interpolation being governed by the time of day, for instance by giving more weight to the prediction of the future circadian phase and/or CBTmin as time progresses.

By way of illustration, FIG. 5B illustrates graphs depicting means and standard deviations of the errors in circadian phase estimation based on using varying preceding periods to determine future light exposure. The errors may be determined in hind-sight, after light exposure measurements for, e.g., an entire week have been gathered. For the circadian phase estimations in FIG. 5B, interpolation is used as described elsewhere. The first prediction of a particular day occurs at approximately 8:00 AM. The first set of means and standard deviations (of the error between predicted and actual circadian phase) corresponds to a preceding period of the preceding 24 hours. As the day progresses, the mean and standard deviation of the error become smaller. The second set of means and standard deviations corresponds to a preceding period of the preceding 3 days. As the day progresses, the mean and standard deviation of the error become smaller. Note that the error using a 1-day preceding period is generally larger than the error using a 3-day preceding period. The third set of means and standard deviations corresponds to a preceding period of the preceding 7 days. As the day progresses, the mean and standard deviation of the error become smaller. Note that the error using a 7-day preceding period is generally smaller than the errors using a 1-day or 3-day preceding period. In other words, FIG. 5B reflects that predicting a future light exposure based on light exposure measurements spanning more preceding days may result in more accurate predictions for the circadian phase of a particular subject. Compared to FIG. 5A, using interpolation as described may improve predictions of future light exposure and the circadian phase of a particular subject.

Power source 72 provides the power to operate one or more components of system 10. Power source 72 may include a portable source of power (e.g., a battery, a fuel cell, etc.), and/or a non-portable source of power (e.g., a wall socket, a large generator, etc.). In one embodiment, power source 72 includes a portable power source that is rechargeable. In one embodiment, power source 72 includes both a portable and non-portable source of power, and the subject is able to select which source of power should be used to provide power to system 10.

Electronic storage 74 includes electronic storage media that electronically store information. The electronic storage media of electronic storage 74 may include one or both of system storage that is provided integrally (i.e., substantially non-removable) with system 10 and/or removable storage that is removably connectable to system 10 via, for example, a port (e.g., a USB port, a FireWire port, etc.) or a drive (e.g., a disk drive, etc.). Electronic storage 74 may include one or more of optically readable storage media (e.g., optical disks, etc.), magnetically readable storage media (e.g., magnetic tape, magnetic hard drive, floppy drive, etc.), electrical charge-based storage media (e.g., EPROM, EEPROM, RAM, etc.), solid-state storage media (e.g., flash drive, etc.), and/or other electronically readable storage media. Electronic storage 74 may store software algorithms, information determined by processor 110, information received via user interface 76, and/or other information that enables system 10 to function properly. For example, electronic storage 74 may record or store one or more illumination parameters (as discussed elsewhere herein), and/or other information. Electronic storage 74 may be a separate component within system 10, or electronic storage 74 may be provided integrally with one or more other components of system 10 (e.g., processor 110).

User interface 76 is configured to provide an interface between system 10 and a user (or medical professional, or other device, or other system) through which the user can provide and/or receive information. This enables data, results, and/or instructions and any other communicable items, collectively referred to as “information,” to be communicated between the user and system 10. An example of information that may be conveyed to a subject is the current time, a scheduled wake-up time, or a scheduled light therapy/treatment. Other examples of information that may be conveyed are: circadian rhythm related information like phase and/or intensity, or user performance related information like scheduled physical or mental performance events. Examples of interface devices suitable for inclusion in user interface 76 include a keypad, buttons, switches, a keyboard, knobs, levers, a display screen, a touch screen, speakers, a microphone, an indicator light, an audible alarm, and a printer. Information may be provided to the subject by user interface 76 in the form of auditory signals, visual signals, tactile signals, and/or other sensory signals.

By way of non-limiting example, user interface 76 may include a light source capable of emitting light. The light source may include, for example, one or more of at least one LED, at least one light bulb, a display screen, and/or other sources. User interface 76 may control the light source to emit light in a manner that conveys to the subject information related to operation of system 10. Note that subject 106 and the user of system 10 may be one and the same person.

It is to be understood that other communication techniques, either hard-wired or wireless, are also contemplated herein as user interface 76. For example, in one embodiment, user interface 76 may be integrated with a removable storage interface provided by electronic storage 74. In this example, information is loaded into system 10 from removable storage (e.g., a smart card, a flash drive, a removable disk, etc.) that enables the user(s) to customize the implementation of system 10. Other exemplary input devices and techniques adapted for use with system 10 as user interface 76 include, but are not limited to, an RS-232 port, RF link, an IR link, modem (telephone, cable, Ethernet, internet or other). In short, any technique for communicating information with system 10 is contemplated as user interface 76

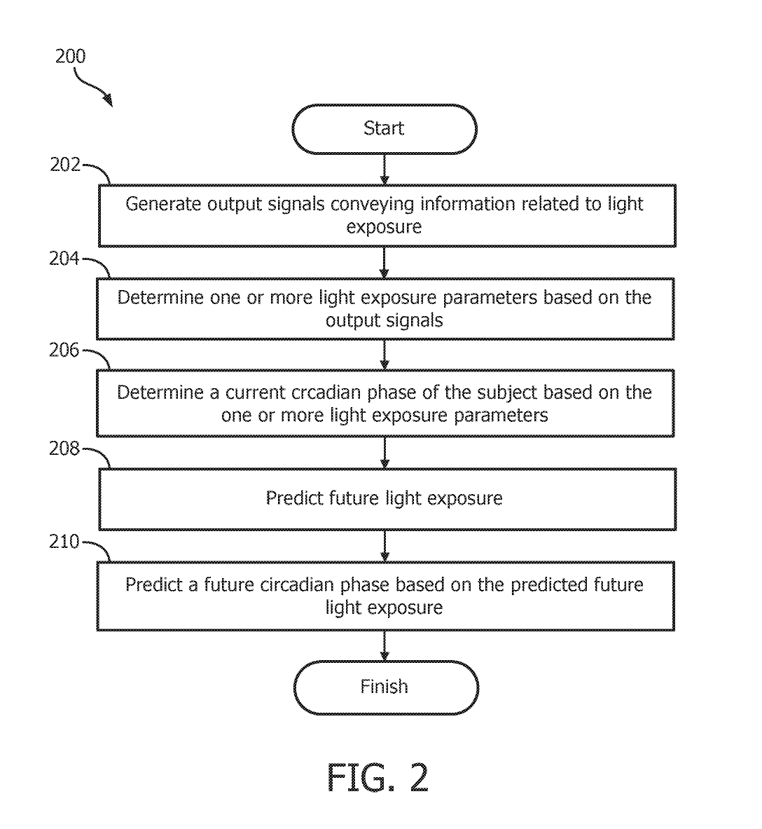

FIG. 2 illustrates a method 200 for predicting circadian phase of subject 106. The operations of method 200 presented below are intended to be illustrative. In some embodiments, method 200 may be accomplished with one or more additional operations not described, and/or without one or more of the operations discussed. Additionally, the order in which the operations of method 200 are illustrated in FIG. 2 and described below is not intended to be limiting.

In some embodiments, method 200 may be implemented in one or more processing devices (e.g., a digital processor, an analog processor, a digital circuit designed to process information, an analog circuit designed to process information, and/or other mechanisms for electronically processing information). The one or more processing devices may include one or more devices executing some or all of the operations of method 200 in response to instructions stored electronically on an electronic storage medium. The one or more processing devices may include one or more devices configured through hardware, firmware, and/or software to be specifically designed for execution of one or more of the operations of method 200.

At an operation 202, output signals conveying information related to light exposure of the subject are generated. In some embodiments, operation 202 is performed by one or more sensors the same as or similar to sensors 142 (shown in FIG. 1 and described herein).

At an operation 204, one or more light exposure parameters are determined based on the generated output signal. In some embodiments, operation 204 is performed by a parameter determination component the same as or similar to parameter determination component 111 (shown in FIG. 1 and described herein).

At an operation 206, a current circadian phase of the subject is determined based on the one or more light exposure parameters. In some embodiments, operation 206 is performed by a phase component the same as or similar to phase component 112 (shown in FIG. 1 and described herein).

At an operation 208, future light exposure is predicted for a first future period based on the generated output signals for a preceding period. In some embodiments, operation 208 is performed by a light exposure component the same as or similar to light exposure component 113 (shown in FIG. 1 and described herein).

At an operation 210, a future circadian phase is predicted. The prediction may be specific with regard to at a future point in time and/or within the first future period. The future circadian phase is based on the current circadian phase and the predicted future light exposure. In some embodiments, operation 210 is performed by a future phase component the same as or similar to future phase component 115 (shown in FIG. 1 and described herein).

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the embodiments have been described in detail for the purpose of illustration based on what is currently considered to be most practical and preferred, it is to be understood that such detail is solely for that purpose and that the disclosure is not limited to the disclosed embodiments, but, on the contrary, is intended to cover modifications and equivalent arrangements that are within the spirit and scope of the appended claims. For example, it is to be understood that the present disclosure contemplates that, to the extent possible, one or more features of any embodiment can be combined with one or more features of any other embodiment.

1. A system to predict circadian phase of a subject, the system comprising:
one or more sensors configured to generate output signals conveying information related to an amount of light exposure of the subject; and
one or more physical processors configured to:
determine one or more light exposure parameters based on the generated output signal;
determine a current circadian phase of the subject based on the one or more light exposure parameters;
predict future light exposure for a first future period based on the generated output signals for a preceding period; and
predict a future circadian phase based on the current circadian phase and the predicted future light exposure.

2. The system of claim 1, wherein the current circadian phase is determined based on a determination of timing of a minimum core body temperature.

3. The system of claim 1, wherein the current circadian phase is determined based on a determination of a dim-light melatonin onset (DLMO).

4. The system of claim 1, wherein the preceding period is selected between about 24 hours and about 28 days.

5. The system of claim 4, wherein the preceding period is selected from a set of options, wherein the one or more physical processors are further configured to determine, per day of the week as the first future period, which preceding period from the set of options most accurately predicted future light exposure, and wherein the preceding period that is used to predict future light exposure is selected based on the day of the week.

6. A method to predict circadian phase of a subject, the method comprising:
generating output signals conveying information related to an amount of light exposure of the subject;
determining one or more light exposure parameters based on the generated output signal;
determining a current circadian phase of the subject based on the one or more light exposure parameters;
predicting future light exposure for a first future period based on the generated output signals for a preceding period; and
predicting a future circadian phase based on the current circadian phase and the predicted future light exposure.

7. The method of claim 6, wherein determining the current circadian phase is based a determination of timing of a minimum core body temperature.

8. The method of claim 6, wherein determining the current circadian phase is based on a determination of a dim-light melatonin onset (DLMO).

9. The method of claim 6, wherein the preceding period is selected between about 24 hours and about 28 days.

10. The method of claim 9, further comprising:
selecting the preceding period from a set of options;
the method further comprising:
determining, per day of the week as the first future period, which preceding period from the set of options most accurately predicted future light exposure, wherein predicting future light exposure includes selecting the preceding period based on the day of the week.

11. A system configured to predict circadian phase of a subject, the system comprising:
means for generating output signals conveying information related to an amount of light exposure of the subject;
means for determining one or more light exposure parameters based on the generated output signal;
means determining a current circadian phase of the subject based on the one or more light exposure parameters;
means for predicting future light exposure for a first future period based on the generated output signals for a preceding period; and
means for predicting a future circadian phase based on the current circadian phase and the predicted future light exposure.

12. The system of claim 11, wherein operation of the means for determining the current circadian phase is based on a determination of a timing of a minimum core body temperature.

13. The system of claim 11, wherein operation of the means for determining the current circadian phase is based on a determination of a dim-light melatonin onset (DLMO).

14. The system of claim 11, further comprising:
means (116 period selection component) for selecting the preceding period, wherein the selected period is between about 24 hours and about 28 days.

15. The system of claim 14, wherein the means for selecting the preceding period is configured to select the preceding period from a set of option, the system further comprising:
a means (117 period accuracy component) for determining, per day of the week as the first future period, which preceding period from the set of options most accurately predicted future light exposure, wherein the means for predicting future light exposure is further configured to select the preceding period based on the day of the week.
